(19) United States
(12) Patent Application Publication
(10) Pub. No.: US 2007/0185322 A1
(43) Pub. Date: Aug. 9, 2007

(54) Methods of extracting RNA

(75) Inventor: Hashem Akhavan-Tafti, Howell, MI (US)

Correspondence Address: RICHARD HANDLEY;Lumigen, Inc., 22900 W. Eight Mile Road, Southfield, MI, 48034, US

(73) Assignee: Nexgen Diagnostics LLC

(21) Appl. No.: 11/703,459

(22) Filed: Feb. 7, 2007

Related U.S. Application Data
(60) Provisional application No. 60/771,510, filed on Feb. 8, 2006.

(51) Int. Cl. C07H 21/02

(52) U.S. Cl. 536/25.4

(57) ABSTRACT

Methods and materials are disclosed for rapid and simple extraction and isolation of RNA from a biological sample involving the use of an acidic solution and a solid phase binding material that has the ability to liberate nucleic acids from biological samples, including whole blood, without first performing any preliminary lysis to disrupt cells or viruses. No detergents or chaotropic substances for lysing cells or viruses are needed or used. Viral, bacterial and mammalian genomic RNA can be isolated using the method of the invention. RNA isolated by the present method is suitable for use in downstream processes such as RT-PCR.

### CROSS REFERENCE TO RELATED APPLICATION

The present application is a continuation in part of co-pending U.S. Provisional Application No. 60/771,510, filed on Feb. 8, 2006.

### FIELD OF THE INVENTION

The present invention relates to materials useful in simplified methods for capturing and extracting ribonucleic acids, particularly ribonucleic acids from materials of biological origin.

### BACKGROUND OF THE INVENTION

Modern molecular biology methods as applied to clinical research, clinical diagnostic testing, and drug discovery have made increasing use of the study of ribonucleic acid (RNA). RNA is present as messenger RNA (mRNA), transfer RNA (tRNA) and ribosomal RNA (rRNA). Several modern molecular biology techniques such as northern blotting, ribonuclease protection assays and RT-PCR require that pure, undegraded RNA be isolated before analysis. Studies of the presence of particular mRNA sequences and levels of expression of mRNAs have become prevalent. Analysis of mRNA, especially using microarrays, is a very powerful tool in molecular biology research. By measuring the levels of mRNA sequences in a sample, the up- or down-regulation of individual genes is determined. Levels of mRNA can be assessed as a function of external stimuli or disease state. For example, changes in p53 mRNA levels have been positively associated with cancer in multiple cell types.

Additionally, a number of viruses with a significant impact on human health, including HIV, HCV, West Nile Virus, Equine Encephalitis Virus, and Ebola Virus have RNA genomes. The ability to rapidly and cleanly extract viral RNA from bodily fluids or tissues is important in virology research and infectious disease diagnostics and treatment.

Current methods for extracting RNA begin with one of a variety of techniques to disrupt or lyse cells, liberate RNA into solution, and protect RNA from degradation by endogenous RNases. Lysis liberates RNA along with DNA and protein from which the RNA must then be separated. Thereafter, the RNA is treated either to solubilize it or to precipitate it. The use of chaotropic guanidinium salts to simultaneously lyse cells, solubilize RNA and inhibit RNases was disclosed in Chirgwin et al, Biochem., 18, 5294-5299 (1979). Other methods separate solubilized RNA from protein and DNA by extraction with phenol/chloroform at low pH (D. M. Wallace, Meth. Enzym., 15, 33-41 (1987)). A commonly used one-step isolation of RNA involves treating cells sequentially with 4 M guanidinium salt, sodium acetate (pH 4), phenol, and chloroform/isoamyl alcohol. Samples are centrifuged and RNA is precipitated from the upper layer by the addition of alcohol (P. Chomczynski, Anal. Biochem., 162, 156-159 (1987)). U.S. Pat. No. 4,843,155 describes a method in which a stable mixture of phenol and guanidinium salt at an acidic pH is added to the cells. After phase separation with chloroform, the RNA in the aqueous phase is recovered by precipitation with an alcohol.

Other methods include adding hot phenol to a cell suspension, followed by alcohol precipitation (T. Maniatis et al, Molecular Cloning, A Laboratory Manual, Cold Spring Harbor Laboratory (1982)); the use of anionic or non-ionic surfactants to lyse cells and liberate cytoplasmic RNA; and the use of inhibitors of RNases such as vanadyl riboside complexes and diethylpyrocarbonate [L. G. Davis et al, “Guanidine Isothiocyanate Preparation of Total RNA” and “RNA Preparation: Mini Method” in Basic Methods in Molecular Biology, Elsevier, New York, pp. 130-138 (1991).

A technique for isolating both DNA and RNA from biological sources by binding on glass or other solid phases was disclosed in U.S. Pat. No. 5,234,809 (Boom et al.). Cells present in biological sources, such as serum or urine, were lysed by exposure to strong (>5 M) solutions of guanidinium thiocyanate in Tris HCl (pH 8.0), containing EDTA and the surfactant Triton X-100. DNA and RNA were purified from biological materials by incubation with diatomaceous earth or silica particles, which formed reversible complexes with the DNA and RNA.

U.S. Pat. No. 5,155,018 to Gillespie provides a process for isolating and purifying biologically active RNA from a biological source, which may also include DNA, proteins, carbohydrates and other cellular materials. RNA is isolated by contacting the biological source with finely divided glass or diatomaceous earth in the presence of a binding solution comprising concentrated, acidified chaotropic salt. Under these conditions, it is claimed that RNA binds selectively to the particulate siliceous material although subsequent treatment of the solid material with ethanolic salt solution to remove DNA is also disclosed. Subsequent work by other investigators have confirmed that contamination with DNA does occur. The RNA which is bound to the particles can be easily separated from the other biological substances contained in the sample. Preferably, the particle-bound RNA is washed to remove non-specifically adsorbed materials. The bound RNA is released from the particles by elution with a dilute salt buffer, and the substantially pure, biologically active RNA is recovered. Addition of a nuclease to destroy DNA in the eluent is also disclosed, calling into further question the claim of selective binding of RNA. U.S. Pat. No. 5,990,302 to Kuroita et al. presents a variation of the Gillespie method for isolating RNA by combining a sample, a chaotrope, a Li salt, an acidic solution and a nucleic acid carrier. U.S. Pat. No. 6,218,531 to Ekenberg provides another improvement wherein the solution containing the RNA and contaminants is mixed with a dilution buffer to form a cleared lysate prior to binding the RNA to a silica solid phase. The clearing is effected by precipitating DNA and proteins. The dilution buffer can be water, but is more preferably a buffer such as SSC having a neutral pH and contains a salt, and more preferably contains a detergent such as SDS.

The ability of singly charged monomeric cationic surfactants to lyse cells and simultaneously precipitate RNA and DNA from solution was described in U.S. Pat. Nos. 5,010,183 and 5,985,572. In these patents RNA is first rendered insoluble. In the method of the '183 patent, a solution of the quaternary ammonium surfactant together with 40% urea and other additives is added to a cell suspension, and the mixture is centrifuged. The pellet is resuspended in ethanol, from which nucleic acids are precipitated by addition of a salt.

U.S. Pat. No. 6,355,792 to Michelsen et al. discloses a method for isolating nucleic acids by acidifying a liquid sample with a buffer having a pH less than 6.5 and contacting the acidic solution with an inorganic oxide material having hydroxyl groups, separating the solid material with bound nucleic acids on it from the liquid, and eluting with alkaline solution having a pH between 7.5 and 11, preferably 8-8.5. The acidic solution is free of ionic detergents, chaotropes and any ions are <0.2 M. The worked examples reflect that use of the method presupposes that nucleic acids have been liberated into solution prior to capture.

WO00/66783 and EP 1206571B1 disclose a method of isolating free, extracellular nucleic acids in a sample by contacting a sample suspected of containing a nucleic acid at a pH of less than 7, with a water-soluble, weakly basic polymer to form a water-insoluble precipitate of the weakly basic polymer with all nucleic acids present in the sample, separating the water-insoluble precipitate from the sample, and contacting the precipitate with a base to raise the solution pH to greater than 7, thereby releasing the nucleic acids from the weakly basic polymer. The polymers contain amine groups that are protonated at acidic pH but neutralized by raising the pH.

U.S. Pat. No. 5,582,988 and EP 0707077 B1 to Backus et al. disclose a method for providing a nucleic acid from a lysate comprising the steps of: at a pH of less than 7, contacting a lysate suspected of containing a nucleic acid with a water-soluble, weakly basic polymer in an amount sufficient to form a water-insoluble precipitate of said weakly basic polymer with all nucleic acids present in said lysate, separating said water-insoluble precipitate from said lysate, and contacting said precipitate with a base to raise the solution pH to greater than 7, and thereby releasing said nucleic acids from said weakly basic polymer.

U.S. Pat. No. 5,973,137 to Heath discloses a method for isolating substantially undegraded RNA from a biological sample by treating the sample with a cell lysis reagent consists of an anionic detergent, a chelating agent and a buffer solution having a pH less than 6. The role of the anionic detergent is said to lyse cells and/or solubilize proteins and lipids as well as to denature proteins. When used to isolate RNA from whole blood, red blood cells are first lysed with a reagent containing NH₄Cl, NaHCO₃ and EDTA, the white blood cells are separated and separately lysed in the presence of a protein-DNA precipitation reagent. The latter is typically a high concentration of a sodium or potassium salt such as acetate or chloride. As a final step, the supernatant containing RNA is precipitated by addition of a lower alcohol. Isolating RNA from yeasts and gram-positive bacteria requires the additional use of a lytic enzyme, glycerol and calcium chloride in order to digest cells in preparation to liberate nucleic acids.

U.S. Pat. No. 5,973,138 to Collis discloses a method for reversible binding of nucleic acids to a suspenion of paramagnetic particles in acidic solution. The particles disclosed in this method were bare iron oxide, iron sulfide or iron chloride. The acidic solution is said to enhance the electropositive nature of the iron portion of the particles and thereby promote binding to the electronegative phosphate groups of the nucleic acids. Related patent U.S. Pat. No. 6,433,160 discloses a similar method wherein the acidic solution contains glycine HCl.

U.S. Pat. No. 6,410,274 to Bhikhabhai discloses a method for purifying plasmid DNA by separating on an insoluble matrix comprising a) lysing cells; b) precipitating most of the chromosomal DNA and RNA with a divalent metal ion; c) removing the precipitate; d) purifying the lysate with an anion exchange resin (using an acidic buffer of pH 4-6, followed by a more alkaline buffer); and e) purifying the plasmid further with a second ion exchange resin.

U.S. Pat. No. 6,737,235 to Cros et al., discloses a method for isolating nucleic acids using particles comprising or coated with a hydrophilic, cross-linked polyacrylamide polymer containing cationic groups. Cationic groups are formed by protonation at low pH of amine groups on the polymer. Nucleic acids are bound in a low ionic strength buffer at low pH and released in a higher ionic strength buffer. The polymers must have a lower critical solubility temperature of 25-45 C. Desorption is also promoted at alkaline pH and higher temperatures.

U.S. Pat. No. 6,875,857 to Simms discloses a method and reagent for isolating RNA from plant material using the reagent composition comprising the nonionic surfactant IGEPAL, EDTA, the anionic surfactant SDS, and a high concentration of 2-mercaptoethanol.

U.S. Pat. No. 7,005,266 to Sprenger-Haussels discloses a method for purifying, stabilizing or isolating nucleic acids from samples containing inhibitors of nucleic acid processing enzymes (e.g. stool) by homogenizing samples and then treating the homogenized sample to form a lysate with a solution having a pH of 2-7, salt concentration >100 mM, and a phenol neutralizing substance such as polyvinylpyrrolidone and, optionally a detergent and a chelating agent. The lysate is then processed on conventional silica-based solid phase materials.

Several patents and applications disclose the reversible capture of nucleic acids onto binding materials mediated by pH change between binding and elution solutions changing the state of protonation of amine groups on the binding materials, e.g U.S. Pat. Nos. 6,270,970; 6,310,199; U.S. Pat. No. 5,652,348; U.S. Pat. No. 5,945,520; W096/09116; WO99/029703; EP 1234832A3; EP 1036082B1; U.S. Application Publication Nos. 2001/0018513, 2003/0008320, and 2003/0054395. Similarly U.S. Pat. No. 6,447,764 to Bayer et al. discloses a method for isolating anionic organic substances, including nucleic acids, from aqueous systems by reversibly binding to non-crosslinked polymer nanoparticles in cationic, protonated form, separating them from the medium, and raising the pH to deprotonate the particles in order to release the anionic organic substance.

U.S. Pat. No. 5,665,582 to Kausch et al. discloses a method for reversibly anchoring a biological material to a solid support comprising placing a reversible polymer onto the solid support, attaching a reversible linker to the polymer, and linking the biological material to the reversible linker with a binding composition, said binding composition comprising a nucleic acid, an antibody, an anti-idiotypic antibody or protein A, to reversibly anchor the biological material to the solid support; wherein said biological material can be a nucleic acid.

U.S. Pat. No. 5,756,126 to Burgoyne discloses a dry solid medium for storage of a sample of genetic material, the medium comprising a solid matrix and a composition sorbed to the matrix, the composition comprising a weak base, a chelating agent and an anionic detergent.

U.S. Pat. No. 6,746,841 to Fomovskaia et al. discloses a method of purifying nucleic acids comprising, in part, providing a dry substrate comprising a solid matrix coated with an anionic surfactant for cellular lysis, applying a sample to the substrate, and capturing nucleic acid. Use for capturing RNA is not specifically disclosed or exemplified.

US 20040014703 to Holländer et al. discloses stabilizing RNA with a composition containing a quaternary ammonium or phosphonium salt compounds and a proton donor such as organic carboxylic acids, ammonium sulfate or phosphoric acid salts at an acidic pH.

GB 2419594 Al discloses stabilizing nucleic acids with amino surfactants and optionally with nonionic surfactants.

U.S. Pat. Nos. 6,602,718; 6,617,170; and 6,821,789; and US Patent Application Publ. 2005/0153292 to Augello disclose methods of preserving biological samples such as whole blood, and preserving RNA and/or DNA by inhibiting or blocking gene induction or nucleic acid degradation. The gene induction blocking agent can comprise a stabilizing agent and an acidic substance. Cationic detergents are preferred stabilizing agents. The latter agents lyse cells and cause precipitation of nucleic acids as a complex with the detergent.

U.S. Pat. No. 6,916,608B2 discloses methods and compositions for stabilizing nucleic acids comprising alcohols and/or ketones in admixture with dimethyl sulfoxide.

U.S. Pat. Nos. 6,204,375 and 6,528,641 disclose methods to stabilize the RNA content of cells by adding to the cells a solution of a salt such as ammonium sulfate at a pH between 4 and 8. The salt solution permeates cells and causes precipitation of RNA along with cellular protein and renders the RNA inaccessible to nucleases which might otherwise degrade it.

The cumbersome multi-step nature of the above methods for isolating RNA complicates the use of RNA in clinical practice. Methods must overcome the difficulty of separating RNA from the protein and DNA in the cell before the RNA is degraded by nucleases, such as RNase. These nucleases are present in blood in sufficient quantities to destroy unprotected RNA rapidly. Successful methods for the isolation of RNA from cells must therefore be capable of preventing degradation by RNases. There remains a need in the art for a rapid, simple method for extracting RNA from biological samples. Such method would minimize hydrolysis and degradation of the RNA so that it can be used in various analyses and downstream processes.

Commonly owned U.S. Patent Application Publication Nos. 2005/0106576, 2005/0106577, 2005/0106589, 2005/0106602, 2005/0136477, and 2006/0234251 disclose materials and methods for extracting nucleic acids, including RNA, from biological materials. The methods rely on a unique class of solid materials for disrupting cells or viruses and do not require a chemical lysis treatment.

### SUMMARY OF THE INVENTION

In one aspect, the present invention provides a novel method for rapid and simple extraction and isolation of RNA from a biological sample involving the use of an acidic solution and a solid phase binding material. Solid phase binding materials used in the practice of the invention have the ability to liberate nucleic acids from biological samples without first performing any preliminary lysis to disrupt cells or viruses. The solid phase binding material can comprise a quaternary ammonium group, a quaternary phosphonium group, or a ternary sulfonium group.

In another aspect, the invention provides a method for extracting and/or purifying RNA from a biological sample involving the use of an acidic solution and a solid phase binding material having a matrix portion and an onium group selected from quaternary ammonium, quaternary phosphonium, and ternary sulfonium groups and further comprising a cleavable linker joining the matrix portion and the onium group.

### DETAILED DESCRIPTION OF THE INVENTION

### Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Biological material—includes whole blood, anticoagulated whole blood, plasma, serum, tissue, cells, cellular content, and viruses.

Cellular material—intact cells or material, including tissue, containing intact cells of animal, plant or bacterial origin. Cells may be intact, actively metabolizing cells, apoptotic cells, or dead cells.

Cellular nucleic acid content—refers to nucleic acid found within cellular material and can be genomic DNA and RNA, and other nucleic acids such as that from infectious materials, including viruses and plasmids.

Magnetic particle—a particle, microparticle, or bead that is responsive to an external magnetic field. The particle may itself be magnetic, paramagnetic or superparamagnetic. It may be attracted to an external magnet or applied magnetic field as when using superparamagnetic or ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Nucleic acid—A polynucleotide can be DNA, RNA or a synthetic DNA analog such as a PNA. Single stranded compounds and double-stranded hybrids of any of these three types of chains are also within the scope of the term.

Release, elute—to remove a substantial portion of a material bound to the surface or pores of a solid phase material by contact with a solution or composition.

RNA—includes, but is not limited to messenger RNA (mRNA), transfer RNA (tRNA) and ribosomal RNA (rRNA).

Sample—A fluid containing or suspected of containing nucleic acids. Typical samples which can be used in the methods of the invention include bodily fluids such as blood, which can be anticoagulated blood as is commonly found in collected blood specimens, plasma, serum, urine, semen, saliva, cell cultures, tissue extracts and the like. Other types of samples include solvents, seawater, industrial water samples, food samples and environmental samples such as soil or water, plant materials, eukaryotes, bacteria, plasmids and viruses, fungi, and cells originated from prokaryotes.

Solid phase material—a material having a surface which can attract nucleic acid molecules. Materials can be in the form of particles, microparticles, nanoparticles, fibers, beads, membranes, filters and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

The present invention is concerned with rapid and simple methods for obtaining RNA from biological samples. The methods utilize a solid phase binding material and an acidic solution into which RNA is released from a source of nucleic acid contained in the sample. The solid phase binding material is selected to have the ability to liberate RNA directly from biological samples without first performing any preliminary lysis to disrupt cells or viruses. Degradation is minimized by liberating the RNA directly into an acidic environment through the action of the solid phase and then rapidly capturing the liberated RNA under acidic conditions onto the solid phase. Moreover, Applicant has discovered that it is possible to recover ribonucleic acids from samples containing RNase activity without the need to resort to the addition of RNase-inactivating compounds or proteins, such as guanidinium salts, high concentration chaotropes or RNase-inhibiting proteins and antibodies.

In practice the method is useful to capture and extract RNA from protein-RNA complexes, intact cells and viruses. RNA can be extracted according to the process of the invention from any biological sample containing nucleic acids, in particular intact cells and viruses. Common sources of these materials include, but are not limited to, bacterial culture or pellets, blood, urine, cells, bodily fluids such as urine, sputum, semen, CSF, blood, plasma, and serum, or from tissue homogenates. The method of the invention can be applied to samples including viable, dead, or apoptotic intact cells and tissues, or cultured bacterial, plant or animal cell lines without the need to subject them to other preliminary procedures. In particular, no preliminary disruption or lysis need be used at all. Extraction of RNA from cells in suspension, i.e., from biological fluids or cell culture, can begin, for example, by pelleting cells with low-speed centrifugation and discarding the medium. RNA may be extracted from intact tissues or organs using tissue disruption methods generally known in the art, for example, by homogenizing, using a hand held homogenizer or an automatic homogenizer, such as a Waring blender, or other tissue homogenizer. The homogenate may be passed through a coarse filter, such as cheesecloth, to remove large particulate matter or the preparation may be centrifuged at low speed to separate particulate material.

The method of this invention is rapid, typically requiring only a few minutes to complete. Significantly, the RNA obtained by the method is of an adequate purity such that it is useful for clinical or other downstream uses, such as the use of reverse transcriptase, by itself or followed by the polymerase chain reaction amplification (RT-PCR), RNA blot analysis and in vitro translation. Advantageously, it is not necessary to isolate cells prior to use of this method and only simple equipment is required for performance of the method. No preliminary lysis or ethanol precipitation step is necessary before processing samples in accordance with the method of the invention. Detergents or chaotropic substances for lysing cells or viruses are not needed or used.

In one embodiment of the present invention, a selected biological sample, containing RNA e.g., a fluid containing cells and/or viruses, is mixed briefly with an acidic solution to form a mixture. The sample and acidic solution need only be in contact in the mixture for as little as a few seconds. No other processing is needed. Either concurrently with or subsequent to the formation of the mixture, the mixture is combined with a solid phase binding material selected to have the ability to liberate RNA directly from biological samples without first performing any preliminary lysis to disrupt cells or viruses. Degradation of RNA is minimized by liberating the RNA directly into an acidic environment through the action of the particles and then rapidly capturing the liberated RNA under acidic conditions onto these particles. The supernatant is removed and the solid phase containing the nucleic acid is optionally washed with one or more wash solutions. If desired, the solid phase can then be eluted to dissociate the RNA from the solid phase. In one embodiment, an alkaline solution is used to elute the RNA from the solid phase or particle. Typically, a desirable concentration of alkali for this purpose is at least 10⁻⁴ M, preferably from about 1 mM to about 1 M.

In another embodiment, the methods of the present invention may, if desired, be performed by the optional use of an RNase inhibitor, such as aurin tricarboxylic acid, DTT, or DEPC. Other inhibitors of RNase may be selected for this purpose by the skilled person.

All of the steps can be performed rapidly, in succession, in a single container or on a single support without the need for specialized equipment such as centrifuges. The method is adaptable to automated platforms for processing large numbers of samples in serial or parallel fashion. All binding and washing steps are preferably done for only a brief period, preferably not more than one minute. Wash steps can preferably be performed in under 10 seconds. Elution is preferably performed in not more than one minute. In an exemplary procedure a 100 μL sample containing a source of RNA is mixed with 100 μL of an acidic solution in a 1.5 mL microcentrifuge tube and briefly mixed by vortexing. Magnetic binding microparticles in an acidic solution are then added and the mixture vortexed for 30 seconds. The supernatant is separated from the particles on a magnetic rack. Particles are washed twice with 200 μL of acidic solution and twice with 200 μL of water. Washed particles are vortex mixed for one minute in alkaline eluent to elute the RNA.

### Solid Phase Materials

In one embodiment, the RNA extraction methods of the present invention utilize a solid phase binding material to rapidly bind the RNA, thereby allowing separation of the RNA from other sample components. The solid phase binding material is selected to have the ability to liberate nucleic acids directly from biological samples without first performing any preliminary lysis to disrupt cells or viruses. The materials for binding nucleic acids in the methods of the present invention comprise a matrix which defines its size, shape, porosity, and mechanical properties. The matrix can be in the form of particles, microparticles, fibers, beads, membranes, and other supports such as test tubes and microwells. Numerous specific materials and their preparation are described in Applicant's co-pending U.S. Applications Publication Nos. 2005/0106576, 2005/0106577, 2005/0106589, 2005/0106602, 2005/0136477, and 2006/0234251.

In one embodiment the materials further comprise a covalently linked nucleic acid binding portion at or near the surface which permits capture and binding of nucleic acid molecules of varying lengths. By surface is meant not only the external periphery of the solid phase material but also the surface of any accessible porous regions within the solid phase material.

In another embodiment the materials further comprise a non-covalently associated nucleic acid binding portion at or near the surface which permits capture and binding of nucleic acid molecules of varying lengths. The non-covalently associated nucleic acid binding portion is associated with the solid matrix by electrostatic attraction to an oppositely charged residue on the surface or is associated by hydrophobic attraction with the surface.

The matrix of these materials carrying covalently or non-covalently attached nucleic acid binding groups can be any suitable substance. Preferred matrix materials are selected from silica, glass, insoluble synthetic polymers, insoluble polysaccharides, and metallic materials selected from metals, metal oxides, and metal sulfides as well as magnetically responsive materials coated with silica, glass, synthetic polymers, or insoluble polysaccharides. Exemplary materials include silica-based materials coated or functionalized with covalently attached surface functional groups that serve to disrupt cells and attract nucleic acids. Also included are suitably surface functionalized carbohydrate based materials, and polymeric materials having this surface functionality. The surface functional groups serving as nucleic acid binding groups include any groups capable of disrupting cells' structural integrity, and causing attraction of nucleic acid to the solid support. Such groups include, without limitation, hydroxyl, silanol, carboxyl, amino, ammonium, quaternary ammonium and phosphonium salts and ternary sulfonium salt type materials described below. Of these, materials having quaternary ammonium, quaternary phosphonium or ternary sulfonium salt groups are preferred.

For many applications it is preferred that the solid phase material be in the form of particles. Preferably the particles are of a size less than about 50 μm and more preferably less than about 10 μm. Small particles are more readily dispersed in solution and have higher surface/volume ratios. Larger particles and beads can also be useful in methods where gravitational settling or centrifugation are employed. Mixtures of two or more different sized particles may be advantageous in some uses.

The solid phase preferably can further comprise a magnetically responsive portion that will usually be in the form of paramagnetic or superparamagnetic microparticles. The magnetically responsive portion permits attraction and manipulation by a magnetic field. Such magnetic microparticles typically comprise a magnetic metal oxide or metal sulfide core, which is generally surrounded by an adsorptively or covalently bound layer to shield the magnetic component. Nucleic acid binding groups can be covalently bound to this layer thereby coating the surface. The magnetic metal oxide core is preferably iron oxide or iron sulfide, wherein iron is Fe²⁺ or Fe³⁺ or both. Magnetic particles enclosed within an organic polymeric layer are disclosed, e.g., in U.S. Pat. Nos. 4,654,267, 5,411,730, and 5,091,206 and in a publication (Tetrahedron Lett.,40 (1999), 8137-8140). Coated magnetic particles are commercially available with several different types of shells. The shells are functionalized as taught in the disclosure of U.S. Patent Application Publication Nos. 2005/0106576, 2005/0106577, 2005/0106589, 2005/0106602, 2005/0136477, and 2006/0234251.

Commercially available magnetic silica or magnetic polymeric particles can be used as the starting materials in preparing magnetic solid phase binding materials useful in the present invention. Suitable types of polymeric particles having surface carboxyl groups are known by the trade names SeraMag™ (Seradyn) and BioMag™ (Polysciences and Bangs Laboratories). A suitable type of silica magnetic particles is known by the trade name MagneSil™ (Promega). Silica magnetic particles having carboxy or amino groups at the surface are available from Chemicell GmbH (Berlin).

Linker groups containing at one terminus a trialkoxysilane group can be attached to the surface of metallic materials or coated metallic materials such as silica or glass-coated magnetic particles. Preferred trialkoxysilane compounds have the formula R¹—Si(OR)₃, wherein R is lower alkyl and R¹ is an organic group selected from straight chains, branched chains and rings and comprises from 1 to 100 atoms. The atoms are preferably selected from C, H, B, N, O, S, Si, P, halogens and alkali metals. Representative R¹ groups are 3-aminopropyl, 2 cyanoethyl and 2-carboxyethyl, as well as groups containing cleavable moieties as described more fully below. In a preferred embodiment, a trialkoxysilane compound comprises a cleavable central portion and a reactive group terminal portion, wherein the reactive group can be converted in one step to a quaternary or ternary onium salt by reaction with a tertiary amine, a tertiary phosphine or an organic sulfide.

It has been found that such linker groups can be installed on the surface of metallic particles and glass or silica-coated metallic particles in a process using fluoride ion. The reaction can be performed in organic solvents including the lower alcohols and aromatic solvents including toluene. Suitable fluoride sources have appreciable solubility in such organic solvents and include cesium fluoride and tetraalkylammonium fluoride salts.

The nucleic acid binding (NAB) groups contained in some of the solid phase binding materials useful in the methods of the present invention may serve dual purposes. NAB groups attract and bind nucleic acids, polynucleotides and oligonucleotides of various lengths and base compositions or sequences. They may also serve in some capacity to free nucleic acid from the cellular envelope. Nucleic acid binding groups include, for example, carboxyl, amine and ternary or quaternary onium groups or mixtures of more than one of these groups. Amine groups can be NH₂, alkylamine, and dialkylamine groups. Preferred nucleic acid binding groups are ternary or quaternary onium groups (-QR₂⁺ or -QR₃⁺) including quaternary trialkylammonium groups (—NR₃⁺), phosphonium groups (—PR₃⁺) including trialkylphosphonium or triarylphosphonium or mixed alkyl aryl phosphonium groups, and ternary sulfonium groups (—SR₂⁺). The solid phase can contain more than one kind of nucleic acid binding group as described herein. Mixtures of more than one size of particles can be used. Mixtures of the above solid phase binding materials with various other solid phase materials with or without NAB groups can also be used. Solid phase materials containing ternary or quaternary onium groups (QR₂⁺ or QR₃⁺) wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Most known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

Preferred embodiments employ solid phase binding materials in which the nucleic acid binding groups are attached to the matrix through a selectively cleavable linkage. Breaking the link effectively “disconnects” any bound nucleic acids from the solid phase. The link can be cleaved by any chemical, enzymatic, photochemical or other means that specifically breaks bond(s) in the cleavable linker but does not also destroy the nucleic acids of interest. Such cleavable solid phase materials comprise a solid support portion comprising a matrix as described above. A nucleic acid binding (NAB) portion for attracting and binding nucleic acids is attached to a surface of the solid support by a cleavable linker portion. Suitable materials with cleavable linkages are described in U.S. Patent Application Publication Nos. 2005/0106576, 2005/0106577, 2005/0106589, 2005/0106602, 2005/0136477, and 2006/0234251, the disclosures of which are incorporated herein by reference.

The cleavable linker portion is preferably an organic group selected from straight chains, branched chains and rings and comprises from 1 to 100 atoms. The atoms are preferably selected from C, H, B, N, O, S, Si, P, halogens and alkali metals. An exemplary linker group is a hydrolytically cleavable group. Examples include carboxylic esters and anhydrides, thioesters, carbonate esters, thiocarbonate esters, urethanes, imides, sulfonamides, sulfonimides and sulfonate esters. In a preferred embodiment the cleavable link is treated with an aqueous alkaline solution. Another exemplary class of linker groups are those groups which undergo reductive cleavage such as a disulfide (S—S) bond which is cleaved by various agents including phosphines and thiols such as ethanethiol, mercaptoethanol, and DTT. Another representative group is an organic group containing a peroxide (O—O) bond. Peroxide bonds can be cleaved by thiols, amines and phosphines. Another representative cleavable group is an enzymatically cleavable linker group. Exemplary groups include esters, which are cleaved by esterases and hydrolases, amides and peptides, which are cleaved by proteases and peptidases, glycoside groups, which are cleaved by glycosidases. Another representative cleavable group is a cleavable 1,2-dioxetane moiety. Such materials contain a dioxetane moiety, which can be decomposed thermally or triggered to fragment by a chemical or enzymatic reagent. Removal of a protecting group to generate an oxyanion promotes decomposition of the dioxetane ring. Fragmentation occurs by cleavage of the peroxidic O—O bond as well as the C—C bond according to a well known process. Cleavable dioxetanes are described in numerous patents and publications. Representative examples include U.S. Pat. Nos. 4,952,707, 5,707,559, 5,578,253, 6,036,892, 6,228,653 and 6,461,876.

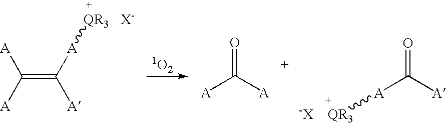

Another cleavable linker group is an electron-rich C—C double bond which can be converted to an unstable 1,2 dioxetane moiety. At least one of the substituents on the double bond is attached to the double bond by means of an O, S, or N atom. Reaction of electron-rich double bonds with singlet oxygen produces an unstable 1,2-dioxetane ring group which rapidly fragments at ambient temperatures to generate two carbonyl fragments.

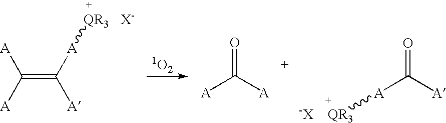

Another group of solid phase materials having a cleavable linker group have as the cleavable moiety a ketene dithioacetal as disclosed in U.S. Pat. Nos. 6,858,733 and 6,872,828. Ketene dithioacetals undergo oxidative cleavage of a double bond by enzymatic oxidation with a peroxidase enzyme and hydrogen peroxide.

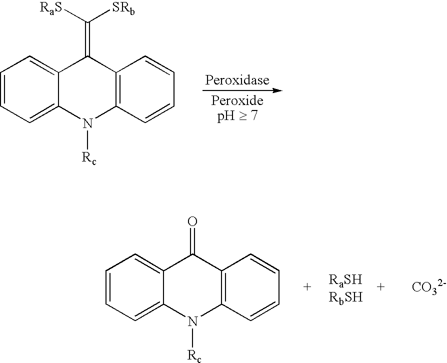

The cleavable moiety can have the structure shown, including analogs having substitution on the acridan ring, wherein Rₐ, R_{b} and R_{c} are each organic groups containing from 1 to about 50 non-hydrogen atoms selected from C, N, O, S, P, Si and halogen atoms and wherein Rₐ and R_{b} can be joined together to form a ring. Numerous other cleavable groups will be apparent to the skilled artisan. Another group of solid phase materials having a cleavable linker group have a photocleavable linker group such as nitro-substituted aromatic ethers and esters. Ortho-nitrobenzyl esters are cleaved by ultraviolet light according to a well-known reaction.

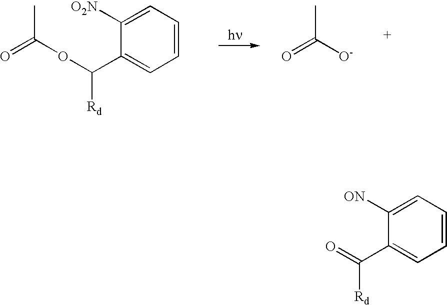

### Acidic Solutions

The acidic solutions used in the methods of the present invention generally encompass any aqueous solution having a pH below neutral pH Preferably the solution will have a pH in the range of 1-5 and more preferably from about 2-4. The acid can be organic or inorganic. Mineral acids such as hydrochloric acid, sulfuric acid, and perchloric acid are useful. Organic acids including monocarboxylic acids, dicarboxylic acids, tricarboxylic acids, and amino acids can be used, as well as salts of the acids. Representative acids include, formic, acetic, trifluoroacetic, propionic, oxalic, malonic, succinic, glutaric, and citric acids, glycine, and alanine. Salts can have any water-soluble counter ion, preferably alkali metal or alkaline earth ions. Acidic solutions comprising salts of transition metals are also useful in the practice of the present invention. Preferred transition metals include Fe, Mn, Co, Cu, and Zn salts.

Unlike other methods employed to extract RNA by chemical lysis, the acidic solutions used in the present method do not contain detergents or chemical lytic agents such as chaotropic substances, e.g guanidinium salts. No organic solvent functioning in either of these capacities, such as DMF or DMSO, is used. The acidic medium, in the absence of other soluble additives, in combination with the solid phase binding material, is sufficient to permit the extraction of intact RNA from the sample, even samples containing RNase enzymes.

The sample and the acidic solution can be mixed together concurrent with the step of combining the mixture with the solid phase by providing the solid phase in the acidic solution. Alternatively the sample may be first mixed together with the acidic solution to form a mixture before combining the mixture with the solid phase.

### Wash Solutions

The wash solution(s) useful in the practice of the present invention, if used, can assist in removing other components from the bound RNA. In one embodiment, a wash solution can comprise the same or a similar acidic solution as was used in the binding step. It has been found advantageous to wash with acidic solutions, possibly in order to remove residual RNase activity. Further washes with water or buffers of neutral pH can be used to neutralize the acid before elution. Water and buffers should be prepared or treated to ensure that they do not have RNase activity.

### Elution Reagents

In one embodiment, the bound RNA is eluted from the solid phase by contacting the solid phase material with a reagent to release the bound RNA into solution. The solution should dissolve and sufficiently preserve the released RNA. RNA eluted in the release solution should be compatible with downstream molecular biology processes. In another embodiment the reagent for releasing the nucleic acid from the solid phase binding material does so by cleavage of a cleavable linker group present in the solid phase binding material. A preferred reagent is a strongly alkaline aqueous solution of at least 10⁻⁴ M. Solutions of alkali metal hydroxides, ammonium hydroxide, tetraalkylammonium hydroxide, alkali metal carbonates and alkali metal oxides at a concentration of at least ₁₀⁻⁴ M are effective in rapidly cleaving and eluting RNA from the cleaved solid phase. When the cleavable group is a disulfide (S—S) group, the elution/cleavage reagent will contain a disulfide-reducing agent, for example a thiol such as ethanethiol, mercaptoethanol, or DTT. When the cleavable group is a peroxide (O—O) bond, the elution/cleavage reagent will contain a reducing agent, for example a thiol, an amine or a phosphine. When the cleavable group is enzymatically cleavable the elution/cleavage reagent will contain a suitable enzyme. Esters will require an esterase or a hydrolase; an amide or a peptide bond will require a protease or a peptidase; a glycoside group will require a glycosidase. When the cleavable group is a 1,2-dioxetane moiety, the dioxetane can be cleaved thermally and the elution reagent can be an alkaline solution as described above. When the cleavable group is a triggerable 1,2-dioxetane moiety the elution/cleavage reagent will contain a chemical or enzymatic reagent to induce cleavage of the group via removal of a protecting group to generate a destabilizing oxyanion. When the cleavable group is an electron-rich C—C double bond which can be converted to an unstable 1,2 dioxetane, the elution/cleavage reagent will contain a source of singlet oxygen such as a photosensitizing dye. Such dyes as are known in the art to react with visible light and molecular oxygen to produce a singlet excited state of oxygen include e.g. Rose Bengal, Eosin Y, Alizarin Red S, Congo Red, and Orange G, fluorescein dyes, rhodamine dyes, Erythrosin B, chlorophyllin trisodium salt, salts of hemin, hematoporphyrin, Methylene Blue, Crystal Violet, Malachite Green, and fullerenes.

In another embodiment the reagent for releasing the RNA from solid phase binding materials comprising a quaternary onium NAB group are selected from the compositions disclosed in Applicant's co-pending U.S. Patent Application Publication 2005/0106589.

The release step can be performed at room temperature, but any convenient temperature can be used. Elution temperature does not appear to be critical to the success of the present methods of isolating nucleic acids. Ambient temperature is preferred, but elevated temperatures may increase the rate of elution in some cases.

### Kits of the Invention

In another embodiment, kits are provided for performing the methods of the invention. A kit for isolating ribonucleic acid from a sample in accordance with the invention comprises at least one solid phase binding material selected to have the ability to liberate nucleic acids directly from biological samples without first performing any preliminary lysis, and an acidic solution. The solid phase binding materials comprise a matrix which can be in the form of particles, microparticles, magnetic particles, fibers, beads, membranes, and other supports such as test tubes and microwells. The matrix is linked covalently or non-covalently to a nucleic acid binding portion, optionally through a cleavable linker.

The nucleic acid binding portion comprises at least one type of group selected from carboxyl, NH₂, alkylamine, dialkylamine groups, quaternary ammonium groups including trialkylammonium groups, quaternary phosphonium groups including trialkylphosphonium, triarylphosphonium, or mixed alkyl aryl phosphonium groups, and ternary sulfonium groups.

The acidic solutions that comprise one element of the kits of the present invention generally encompass any aqueous solution having a pH below neutral pH Preferably the solution will have a pH in the range of 1-5 and more preferably from about 2-4. The acid can be organic or inorganic. Mineral acids such as hydrochloric acid, sulfuric acid, and perchloric acid are useful. Organic acids including monocarboxylic acids, dicarboxylic acids, tricarboxylic acids, and amino acids can be used, as well as salts of the acids. Representative acids include, formic, acetic, trifluoroacetic, propionic, oxalic, malonic, succinic, glutaric, and citric acids, glycine, and alanine. Salts can have any water-soluble counter ion, preferably alkali metal or alkaline earth ions. Acidic solutions comprising salts of transition metals are also useful in the practice of the present invention. Preferred transition metals include Fe, Mn, Co, Cu, and Zn salts.

Kits may additionally comprise an elution reagent, and one or more optional wash buffers and other conventional components of kits such as instruction manuals, protocols, buffers and diluents. Elution reagents may be selected from strongly alkaline aqueous solutions such as solutions of alkali metal hydroxides or ammonium hydroxide at a concentration of at least 10⁻⁴ M, preferably from about 1 mM to about 1 M, disulfide-reducing agents, such as thiols including ethanethiol, mercaptoethanol, or DTT, peroxide-reducing agents, such as thiols, amines or phosphines, and enzymes such as esterases, hydrolase, proteases, peptidases, glycosidases or peroxidases. In an embodiment wherein a solid phase binding material contains a cleavable linker such as an electron-rich alkene group that is cleavable by reaction with a source of singlet oxygen, the kit may comprise a photosensitizing dye as described above.

### EXAMPLES

### Example 1

### Solid Phase Material Useful in Isolating RNA

Synthesis of magnetic particles functionalized with a tributylphosphonium NAB group and a cleavable arylthioester linkage.

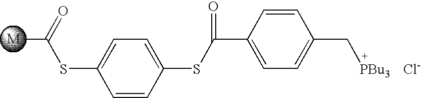

a) Preparation of magnetite. Argon was bubbled through 3 L of type I water in a 5 L flask for one hour. Concentrated NH₄OH (28%, 180 mL) was added under Ar. A mixture of 50 mL of 2 M FeCl₂ in 1 M HCl and 200 mL of 1 M FeCl₃ in 1 M HCl was added via addition funnel over a period of about one hour. The solids were collected in two flasks by pouring 500-600 mL portions into a flask with a disk magnet on the outside, decanting the supernatant each time. The solid was washed by dispersion in 500-600 mL of type I water with sonication followed by attracting to a magnet and decanting the supernatant. The process was repeated until the pH of the supernatant was ca. 8.5. The contents of the two flasks were combined so that the magnetite was stored in a total volume of ca. 500 mL.

b) A 500 mL flask was charged with 3-methylaminopropyltrimethoxysilane (149.8 g) and purged with Ar. After placing the flask in an ice bath, acryloyloxytrimethylsilane (119.6 g) was added slowly via syringe. The reaction was stirred for 5 minutes, the ice bath removed and stirring continued for 2 hours. The product was used without further purification.

c) Coating of magnetite. A quantity of the magnetite slurry from step a) containing 5.0 g of magnetite was diluted to 140 mL with type I water and the mixture sonicated. Ethanol (1.25 L) was added after 15 minutes. Concentrated NH₄0H (28%, 170 mL) was added after 30-45 minutes. A solution of 1.5 g of the silyl ester from step b) and 13.5 g of Si(OEt)₄ in ethanol was added in three portions to the reaction over a period of 90 minutes. A solution of 3.75 g of silyl ester compound in 20-30 mL of ethanol was then added and the mixture stirred and sonicated for an additional 90 minutes. Stirring was maintained over night. The mixture was transferred in 500 mL portions into two 1 L flasks and the particles were separated magnetically. The solids were washed sequentially with 4×250 mL of methanol, 2×250 mL of type I water, 1×250 mL of pH 1 dilute HCl in type I water (for 10 minutes before placing mixture back on magnets), 4×250 mL of type I water, 4×250 mL of methanol, and 2×250 mL of acetone. Solids were air-dried over night. During this step hydrolysis of the silyl ester occurred resulting in the creation of a carboxylic acid group.

d) The magnetic carboxylic acid-functionalized particles from the previous step (1.0 g) were placed in 30 mL of thionyl chloride and refluxed for 4 hours. The excess thionyl chloride was decanted from the magnetic solids. The particles were washed with CH₂Cl₂ several times and taken on to the next step.

e) The acid chloride functionalized particles, suspended in 50 mL of CH₂Cl₂, were treated with 0.22 g of 1,4-benzenedithiol and 0.52 mL of diisopropylethyl amine. The mixture was sonicated for 5 min and agitated with an orbital shaker over night. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solids were air-dried over night.

f) A mixture of the particles of the preceding step (ca. 0.9 g) and 25 mL of CH₂Cl₂ was treated with 0.81 g of tributylphosphine. The mixtures were sonicated for 5 minutes and agitated with an orbital shaker over night. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solids were air-dried over night.

g) A mixture of the particles of the preceding step (ca. 0.8 g) and 25 mL of CH₂Cl₂ was treated with 0.25 g of 4-chloromethylbenzoyl chloride and 0.52 mL of diisopropylethylamine. The mixture was sonicated for 5 min and agitated with an orbital shaker over night. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solid was collected and dried over night.

h) A mixture of the particles of the preceding step (ca. 0.7 g) and 25 mL of CH₂Cl₂ was treated with 0.41 g of tributylphosphine. The mixture was sonicated for 5 min and agitated with an orbital shaker for a total of 7 days. The solids were washed sequentially, using magnetic separation, with 1:1 CH₂Cl₂ /CH₃OH and CH₃0H. Solid was collected and dried.

### Example 2

### Larger Particle Size Solid Phase Material

Synthesis of magnetic particles functionalized with a tributylphosphonium NAB group and a cleavable arylthioester linkage.

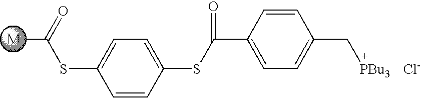

a) A 500 mL flask was charged with 3-methylaminopropyltrimethoxysilane (149.8 g) and purged with Ar. After placing the flask in an ice bath, acryloyloxytrimethylsilane (119.6 g) was added slowly via syringe. The reaction was stirred for 5 minutes, the ice bath removed and stirring continued for 2 hours. The product was used without further purification.

b) Commercial magnetite (Strem cat. No. 93-2616 1-5 μm) 5.0 g was diluted with 140 mL of type I water and 1.25 L of ethanol. Concentrated NH₄OH (28%, 170 mL) was added after 30-45 minutes. A solution of 1.5 g of the silyl ester from step b) and 13.5 g of Si(OEt)₄ in ethanol was added in three portions to the reaction at 90 minute intervals. A solution of 3.75 g of silyl ester compound in 20-30 mL of ethanol was then added and the mixture stirred and sonicated for an additional 90 minutes. Stirring was maintained over night. The mixture was transferred in 500 mL portions into two 1 L flasks and the particles were separated magnetically. The solids were washed sequentially with 4×250 mL of methanol, 2×250 mL of type I water, 1×250 mL of pH 1 dilute HCl in type I water (for 10 minutes before placing mixture back on magnets), 4×250 mL of type I water, 4×250 mL of methanol, and 2×250 mL of acetone. Solids were air-dried over night. During this step hydrolysis of the silyl ester occurred resulting in the creation of a carboxylic acid group.

d) The magnetic carboxylic acid-functionalized particles from the previous step (1.0 g) were placed in 30 mL of thionyl chloride and refluxed for 4 hours. The excess thionyl chloride was decanted from the magnetic solids. The particles were washed with CH₂Cl₂ several times and taken on to the next step.

e) The acid chloride functionalized particles, suspended in 50 mL of CH₂Cl₂, were treated with 0.22 g of 1,4-benzenedithiol and 0.52 mL of diisopropylethyl amine. The mixture was sonicated for 5 min and agitated with an orbital shaker over night. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solids were air-dried over night.

f) A mixture of the particles of the preceding step (ca. 0.9 g) and 25 mL of CH₂Cl₂ was treated with 0.81 g of tributylphosphine. The mixtures were sonicated for 5 minutes and agitated with an orbital shaker over night. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solids were air-dried over night.

g) A mixture of the particles of the preceding step (ca. 0.8 g) and 25 mL of CH₂Cl₂ was treated with 0.25 g of 4-chloromethylbenzoyl chloride and 0.52 mL of diisopropylethylamine. The mixture was sonicated for 5 min and agitated with an orbital shaker over night. The solids were washed sequentially, using magnetic separation, with 1:1 CH₂Cl₂ /CH₃OH and CH₃OH. Solid was collected and dried over night.

h) A mixture of the particles of the preceding step (ca. 0.7 g) and 25 mL of CH₂Cl₂ was treated with 0.41 g of tributylphosphine. The mixture was sonicated for 5 min and agitated with an orbital shaker for a total of 7 days. The solids were washed sequentially, using magnetic separation, with CH₂Cl₂, 1:1 CH₂Cl₂/CH₃OH, CH₃OH, 1:1 CH₂Cl₂/CH₃OH, and CH₂Cl₂. Solid was collected and dried.

### Example 3

### Synthesis of Functionalized Magnetic Polymer

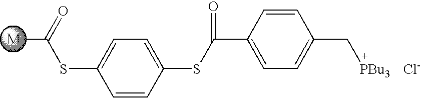

An aliquot of beads (Dynal magnetic COOH beads, Lot No. G36710) containing 25 mg of solid was decanted by the aid of a magnet. Beads were then washed with 3×1 mL of water, and 3×1 mL CH₃CN before drying for 4 hrs. The beads were suspended in 1 mL of CH₂Cl₂ to which was added 28.8 mg of EDC and shaken for 30 min. A solution of 1,4-benzenedithiol (30 mg) was added to the mixture. The tube was sonicated for 1 min and shaken over night. The supernatant was removed and the beads were washed magnetically with 4×1 mL of CH₂Cl₂, 1 mL of 1:1 MeOH: CH₂Cl₂, 4×1 mL of MeOH and 4×1 mL of CH₂Cl₂.

The beads were suspended in 1 mL of CH₂Cl₂to which was added 140 μL of tributylphosphine. The reaction mixture was vortexed for 1 min and shaken for a total of 3 days. The solvent was decanted by keeping on a magnet. Beads were washed magnetically with 4×1 mL of CH₂Cl₂, 1 mL of 1:1 MeOH: CH₂Cl₂, 4×1 mL of MeOH, 1 mL of 1:1 MeOH: CH₂Cl₂, and 4×1 mL of CH₂Cl₂.

A mixture of the particles of the preceding step (ca. 25 mg) in 1 mL of CH₂Cl₂ was treated with 2) mg of 4-chloromethylbenzoyl chloride and 52 μL of diisopropylethylamine. The mixture was vortexed for 10 s, sonicated for 5 min and agitated with an orbital shaker over night. The solids were washed sequentially, using magnetic separation, with 4×1 mL of CH₂Cl₂, 1 mL of 1:1 MeOH: CH₂Cl₂, 4×1 mL of MeOH, 1 mL of 1:1 MeOH: CH₂Cl₂, and 4×1 mL of CH₂Cl₂.

A mixture of the particles of the preceding step (25 mg) and 1 mL of CH₂Cl₂ was treated with 30 mg of tributylphosphine. The mixture was sonicated for 2 min and agitated with an orbital shaker for a total of 6 days. The solids were washed sequentially, using magnetic separation, with 4×1 mL of CH₂Cl₂, 3×1 mL of MeOH, 2×1 mL of water. A stock solution of beads (25 mg/mL) was made by adding 1 mL of water.

### Example 4

### Synthesis of Functionalized Magnetic Polymer

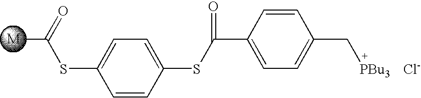

Magnetic particles from 2×0.535 mL of Sera-Mag™ Magnetic Carboxylate-Modified microparticle suspension (Seradyn) (which contains a total of 50 mg of particles) were magnetically collected and the supernatant decanted. Beads were then washed with 3×1 mL of water, 3×1 mL CH₃CN, and 3×1 mL of CH₂Cl₂. The beads were suspended in 3.6 mL of CH₂Cl₂ to which was added 60 mg of EDC and shaken for 30 min.

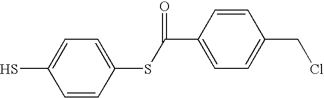

Preparation of linker: 1,4-Benzenedithiol (11.97 g) was dissolved in 300 mL of The solution was cooled to −78° C. A solution of 8.86 g of 4-chloromethylbenzoyl chloride and 3.8 mL of pyridine in 100 mL of CH₂Cl₂ was added dropwise over 1 hour. The reaction solution was allowed to warm to room temperature and maintained over night. After workup 1 g of the impure solid product was washed with either to produce 200 mg of pure product. An additional quantity could be isolated from the filtrate chromatographically.

A solution of linker (60 mg) in 400 μL of DMF was added to the mixture. The tube was sonicated for 1 min and shaken over night. The beads were split into two 25 mg portions and processed separately. The supernatant was removed and the beads were washed magnetically with 4×1 mL of CH₂Cl₂, 1 mL of 1:1 MeOH: CH₂Cl₂, 4×1 mL of MeOH, 1 mL of 1:1 MeOH: CH₂Cl₂, and 4×1 mL of CH₂Cl₂.

The particles were suspended in 10 mL of CH₂Cl₂ to which was added 75 μL of tributylphosphine. The reaction mixture was vortexed for 1 min and shaken for a total of 7 days. The beads were split into two 25 mg portions and processed separately. The solvent was decanted by keeping on a magnet. Beads were washed magnetically with 3×1 mL of CH₂Cl₂, 1 mL of 1:1 MeOH: CH₂Cl₂, 4×1 mL of MeOH, and 2×1 mL of water. A stock solution of beads (25 mg/mL) was made by adding 1 mL of water.

### Example 5

### Acidic Solutions Useful in Extracting RNA

A simple test system was utilized for demonstrating the utility of the present method in recovering RNA and for evaluating the relative efficacy of various conditions and reagents. A mixture of 100 μL of test solution and 100 μL of fetal bovine serum (FBS) was made. Luciferase RNA, 2 μL of 1 μg/μL, was added and the mixture vortex mixed for 1 minute. The mixture was combined with a suspension of 2 mg of the particles of example 1 in 100 μL of test solution and vortex mixed for 30 seconds. The liquid was removed from the particles on a magnetic rack and the particles washed sequentially with 2×200 μL of test solution and 2×200 μL of 0.1% DEPC-treated water. RNA was extracted by sequentially combining the particles with 50 μL of 50 mM NaOH, vortex mixing for 1 minute and removing the eluent. Supernatants from the initial binding reaction were analyzed on ethidium-stained gels and by fluorescent staining to determine the quantity of RNA that had been removed from solution and bound to the particles. Eluents were analyzed on ethidium-stained gels and by fluorescent staining to determine the quantity and quality of the RNA extracted by the procedure. Use of the following solutions led to quantitative binding of RNA, and elution of substantial amounts of the bound RNA.

| Test Solution | pH | Test Solution | pH |
|---|---|---|---|
| Na citrate 0.3 M | 4.0 | Glycine 0.3 M | 3.0 |
| Na citrate 0.3 M | 3.5 | Glycine 0.3 M | 2.5 |
| Na citrate 0.3 M | 3.0 | Glycine 0.05 M | 2.5 |
| Na citrate 0.05 M | 3.0 | Na glutarate 0.3 M | 4.0 |
| K acetate 0.3 M | 4.0 | Na glutarate 0.3 M | 3.2 |
| K acetate 0.05 M | 4.0 | Na succinate 0.3 M | 4.0 |
| K acetate 0.3 M | 3.7 | Na succinate 0.3 M | 3.8 |
| Na acetate 0.3 M | 4.0 | | |

### Example 6

### Extraction of RNA from E. coli Culture

A simple test system was utilized for demonstrating the utility of the present method in recovering RNA from E. coli grown in culture and for evaluating the relative efficacy of various conditions and reagents. A 200 μL portion of E. Coli culture was pelleted and the medium removed. The pellet was combined with 200 μL of test solution and 2 mg of the particles of example 1 and vortex mixed for 30 seconds. The liquid was removed from the particles on a magnetic rack and the particles washed sequentially with 2×200 μL of wash solution and 2×200 μL of 0.1% DEPC-treated water. RNA was isolated by combining the particles with 50 μL of a solution of 50 mM NaOH and 20 mM tris pH 8.8, vortex mixing for 1 minute and removing the solution. Supernatants from the initial binding reaction were analyzed on ethidium-stained gels and by fluorescent staining to determine the quantity of RNA that had been removed from solution and bound to the particles. Eluents were analyzed on ethidium-stained gels and by fluorescent staining to determine the quantity and quality of the RNA extracted by the procedure. Use of the following solutions led to recovery of substantial amounts of intact RNA. In comparison, binding of the pellet and washing the particles in 0.1% DEPC-treated water produced only degraded RNA.

| Test Solution | Wash solution | |
|---|---|---|
| Acetic acid 0.05 M | Na citrate 0.3 M | pH 3 |
| Acetic acid 0.1 M | Na citrate 0.3 M | pH 3 |
| Acetic acid 0.2 M | Na citrate 0.3 M | pH 3 |
| Trifluoroacetic acid 0.05 M | Trifluoroacetic acid 0.05 M | |
| Trifluoroacetic acid 0.1 M | Trifluoroacetic acid 0.1 M | |
| Trifluoroacetic acid 0.2 M | Trifluoroacetic acid 0.2 M | |

### Example 7

### Additional Conditions for Extraction of RNA from E. coli Culture

Performing the isolation of E. coli according to the method of Example 6 with the following test acidic solutions also resulted in producing intact RNA as evidenced by the band pattern in the electrophoresis gel.

Test Solution

| | |
|---|---|
| Zinc acetate 0.05 M + 0.1 M ammonium acetate | pH 4.0 |
| Methyltributylphosphonium methosulfate 0.1 M–1 M | |
| Na succinate 0.0.5 M | pH 3 |

### Example 8

### Extraction of RNA from Armored RNA

Armored RNA® (Ambion Diagnostics, Austin, Tex.) is a protein-encapsidated ssRNA and represents a pseudo-viral particle. An Armored RNA for HIV-B sequence, comprising a segment from the gag region and viral coat proteins, was used to test the methods of the invention for isolating RNA from a complex sample.

A typical procedure for extracting RNA from Armored RNA in plasma is as follows. A 105 μL solution composed of 5 μL of Armored RNA (containing 50,000 copies) in 100 μL of EDTA anti-coagulated plasma (Equitech-Bio, Inc., Kerrville, Tex.) was combined with 100 μL of test solution (e.g. 50 mM KOAc, pH 4.0) and the mixture vortexed briefly to mix. After 1 minute, the mixture was combined with 2 mg of the particles of example 1 in 100 μL of 50 mM KOAc, pH 4.0 and the slurry vortex mixed for 30 seconds. The particles were separated on a magnetic rack and washed sequentially with 2×200 μL of 50 mM KOAc, pH 4.0 and 2×200 μL of 0.1% DEPC-treated water. RNA was eluted by vortex mixing the particles with 50 μL of 50 mM NaOH for 1 minute and removing the solution. Comparisons were made with controls in which 105 μL of plasma/Armored RNA was combined with 2 mg of particles and 200 μL of 0.1% DEPC-treated water in place of the test solution.

RNA-containing eluents were subjected to RT-PCR amplification using a primer set to amplify a segment of the gag gene. Amplification reactions were performed with an iScript™ One-Step RT-PCR Kit with SYBR® Green (Bio-Rad) using an iCycler instrument (Bio-Rad) for amplification and detection.

The following test solutions permitted the recovery of amplifiable RNA as evidenced by C_{T} values significantly lower than the water control.

Test Solution

| K acetate 0.3 M | | pH 4.0 |
|---|---|---|
| K acetate 0.05 M | | pH 4.0 |
| Acetic acid 0.05 M | | |
| Acetic acid 0.2 M | | |
| Trifluoroacetic acid | | 0.05 M |
| Pyridinium HCl | | 0.05 M |
| Hydrochloric acid | | 0.025 M |
| Tetrabutylphosphonium hydrochloride | | 0.05 M |
| K acetate 0.05 M + Acetic acid 0.05 M | | |
| Zinc acetate 0.05 M | | pH 4.0 |
| K acetate 0.05 M, pH 4.0 + | 50:50 | pH 1.8 |
| Trifluoroacetic acid 0.05 M | | |
| | 70:30 | pH 2.1 |
| | 80:20 | pH 2.5 |
| Zinc acetate 0.05 M, pH 4.0 + | (80:20) | |
| Trifluoroacetic acid 0.05 M | | |

| | |
|---|---|
| Mg acetate 0.05 M | pH 4.0 |
| Ammonium acetate 0.05 M | |
| Tetrabutylammonium acetate 0.05 M | |
| Tetraethylammonium acetate 0.05 M | |
| Zinc acetate 0.05 M, pH 4.0 + | |
| Trifluoroacetic acid (pH 2.0, 2.5. 3.0, 3.5) | |
| Zinc acetate 0.05 M + Glycine 0.05 M | pH 3.3 |
| Zinc acetate 0.05 M + Na citrate 0.05 M | pH 3.3 |
| Zinc acetate 0.05 M + Na citrate 0.05 M | pH 4.2 |
| Zinc chloride 0.05 M + Glycine 0.05 M | pH 2.75 |
| Zinc chloride 0.05 M + Na citrate 0.05 M | pH 2.5 |

### Example 9

### Extraction of RNA from Armored RNA

In an alternative method fetal bovine serum (FBS) (Invitrogen, Carlsbad, Calif.) was used in place of plasma. Comparisons were made with controls in which 105 μL of FBS/Armored RNA was combined with 2 mg of particles and 200 μL of 0.1% DEPC-treated water in place of the test solution. RNA-containing eluents were analyzed by RT-PCR as described in example 4. Most of the test solutions of example 4 in addition to those listed below permitted the recovery of amplifiable RNA as evidenced by C_{T} values significantly lower than the water control.

Test Solution

| | |
|---|---|
| Glycine 0.05 M | pH 2.5 |
| Glycine 0.3 M | pH 2.5 |
| Glycine 0.3 M | pH 3.0 |
| Na citrate 0.3 M | pH 3.5 |
| Na citrate 0.1 M | pH 3.5 |
| Na citrate 0.3 M | pH 3.0 |

### Example 10

The procedure of Example 8 for isolating and amplifying Armored RNA added into plasma was performed successfully using each of the solid phase materials of Examples 1, 2, 3, and 4 and with various acidic solutions.

| Solid Phase | Acidic Solution |
|---|---|
| Example 1 | Co acetate 0.05 M, pH 4.0 |
| Example 1 | Mn acetate 0.05 M, pH 4.0 |
| Example 1 | Co acetate 0.05 M + K acetate 0.05 M, pH 4.0 |
| Example 2 | K acetate 0.05 M, pH 4.0 |
| Example 2 | Zn acetate 0.05 M, pH 4.0 |
| Example 3 | Zn acetate 0.05 M, pH 4.0 |
| Example 4 | Zn acetate 0.05 M, pH 4.0 |

### Example 11

### Extraction and Analysis of HIV RNA from Plasma

The methods of the present invention were used for extracting RNA from HIV-positive plasma processed from EDTA-anticoagulated blood. Samples were tested for the presence of HIV RNA using a COBAS AMPLICOR HIV-1 MONITOR TEST ver. 1.5 (Roche Diagnostics). This test is an automated RT-PCR test for quantitating HIV-1 RNA by reverse transcription of RNA into a cDNA copy, PCR amplification of a 155 base pair sequence within the highly conserved region of the gag gene, hybridization of biotin-labeled amplicon to oligonucleotide probes bound to magnetic particles, binding of biotin labels with avidin-horseradish peroxidase conjugate, and colorimetric detection with TMB.

The sample preparation methodology provided with the kit was replaced by one using the present invention as described below.

### Procedure for HIV RNA Extraction

1. A slurry of 2 mg of the particles of example 1 in 100 μL of 50 mM KOAc, pH 4 was prepared.
2. Add 100 μL of 50 mM KOAc, pH 4 to 100 μL of plasma. Touch vortex the mixture and incubate for 1 minute at room temperature.
3. Add plasma solution to the bead slurry and vortex mix the mixture for 30 seconds.
4. Remove supernatant, add 200 μL of 50 mM KOAc, pH 4. Vortex 5 seconds.
5. Repeat step #4.
6. Remove supernatant, add 200 μL of 0.1% DEPC-treated water. Vortex 5 seconds.
7. Repeat step #6.
8. Remove all remaining buffer. Add 50 μL of 50 mM NaOH and vortex for 1 minute.
9. Transfer eluent to a clean 1.5 mL tube.
10. Add 150 μL of 0.1% DEPC-treated water to the particles for a second elution and vortex for 1 minute.
11. Combine the second eluent with the first eluent.
12. Add 50 μL of the combined eluent to the HIV-1 MONITOR Test, ver. 1.5.

After performing the COBAS AMPLICOR amplification, hybridization and immunobinding, serial dilutions were made prior to detection. When using the above protocol, analysis of a plasma specimen known to contain 1.88×10⁵ of HIV particles/mL permitted detection of a 1:729 dilution in the ELISA.

### Example 12

### Extraction of RNA from Human Whole Blood

A simple test system was utilized for demonstrating the utility of the present method in recovering RNA from Human Whole Blood. As a model for freshly drawn blood which would still have intact RNA, cultured Human T-lymphocyte cells (Jurkat) were spiked into whole blood (CPD anti-coagulated) for evaluating the relative efficacy of various conditions and reagents.

7×10⁵ Jurkat cells were pelleted and the medium removed. The pellet was combined with 100 μL of human whole blood. The blood was combined with 100 μL of test solution containing 2 mg of the particles of Example 1 or 2 and vortex mixed for 30 seconds. The liquid was removed from the particles on a magnetic rack and the particles washed sequentially with 2×500 μL of wash solution and 2×500 μL of 0. 1% DEPC-treated water. RNA was isolated by combining the particles with 50 μL of a solution of 50 mM NaOH and 20 mM tris pH 8.0, vortex mixing for 1 minute and removing the solution. The eluent was neutralized with 50 μL of 100 mM Zinc Acetate pH4, and rebound to fresh beads by combining the neutralized eluent with 100 μL of test solution containing 2 mg of the particles of Example 1 or 2 and vortex mixed for 30 seconds. The liquid was removed from the particles on a magnetic rack and the particles washed sequentially with 2×500 μL of wash solution and 2×500 μL of 0.1% DEPC-treated water. RNA was isolated by combining the particles with 50 μL of a solution of 50 mM NaOH and 20 mM tris pH 8.0, vortex mixing for 1 minute and removing the solution.

RNA-containing eluents were subjected to RT-PCR and PCR using primer sets to amplify RNA and DNA of the GAPDH and 1 8S genes. Amplification reactions were performed with an iScript™ One-Step RT-PCR kit with SYBR® Green (Bio-Rad) using an iCycler instrument (Bio-Rad) for amplification and detection. Positive amplification results were obtained (C_{T} for RT-PCR<C_{T} for PCR).

| Solid Phase | Acidic Solution |
|---|---|
| Example 1 | Zn acetate 0.05 M, pH 4.0 |
| Example 1 | 3,3-dimethylglutaric acid 0.05 M, pH 3.2 |
| Example 2 | Zn acetate 0.05 M, pH 4.0 |

What is claimed is:

1. A method for extracting ribonucleic acid from a biological sample containing at least one of cells or viruses comprising:
a) contacting the sample with an acidic solution to form a mixture;
b) combining the mixture with a solid phase binding material selected to have the ability to liberate ribonucleic acid directly from biological samples without first performing any preliminary lysis, and wherein no chaotropic agents or detergents are used to effect lysis, and whereby the solid phase binding material causes lysis of cells and viruses to liberate ribonucleic acid; and
c) binding ribonucleic acid on the solid phase.

2. The method of claim 1 further comprising:
d) separating the sample from the solid phase having ribonucleic acid bound thereto
e) optionally washing the solid phase with at least one wash solution; and
f) eluting the bound ribonucleic acid from the solid phase by contacting the solid phase material with a reagent to release the bound RNA into solution.

3. The method of claim 1 wherein the step of forming the mixture of the sample and the acidic solution is concurrent with the step of combining the mixture with the solid phase.

4. The method of claim 1 wherein the mixture of the sample and the acidic solution is formed before the step of combining the mixture with the solid phase.

5. The method of claim 1 wherein the solid phase is selected from particles, microparticles, fibers, beads, membranes, test tubes and microwells.

6. The method of claim 1 wherein the solid phase comprises a matrix portion and a nucleic acid binding portion.

7. The method of claim 6 wherein the matrix portion is selected from silica, glass, insoluble synthetic polymers, insoluble polysaccharides, metals, metal oxides, and metal sulfides.

8. The method of claim 6 wherein the matrix portion is selected from magnetically responsive materials coated with silica, glass, synthetic polymers, or insoluble polysaccharides.

9. The method of claim 1 wherein the solid phase comprises microparticles having a diameter of less than 10 μm.

10. The method of claim 9 wherein the microparticles are magnetically responsive.

11. The method of claim 9 wherein mixtures of more than one size of particles are used.

12. The method of claim 11 wherein particles of at least one size have a nucleic acid binding portion and particles of at least one other size do not have a nucleic acid binding portion.

13. The method of claim 6 wherein the solid phase material further comprises a covalently linked nucleic acid binding portion which permits capture and binding of ribonucleic acids.

14. The method of claim 1 wherein the solid phase materials further comprise a non-covalently associated nucleic acid binding portion which permits capture and binding of ribonucleic acids.

15. The method of claim 1 wherein solid phase material further comprises a silica-based material functionalized with covalently incorporated surface functional groups that serve to disrupt cells and attract nucleic acids selected from hydroxyl, silanol, carboxyl, amino, ammonium, quaternary ammonium and phosphonium salts and ternary sulfonium salts.

16. The method of claim 1 wherein the solid phase material further comprises a polymeric material having covalently incorporated surface functional groups that serve to disrupt cells and attract nucleic acids selected from hydroxyl, silanol, carboxyl, amino, ammonium, quaternary ammonium and phosphonium salts and ternary sulfonium salts

17. The method of claim 13 wherein the nucleic acid binding portion is comprised of a plurality of nucleic acid binding groups selected from carboxyl, NH₂, alkylamine, and dialkylamine groups, ternary or quaternary onium groups or mixtures of more than one of these groups.

18. The method of claim 17 wherein the nucleic acid binding portion is comprised of a plurality of nucleic acid binding groups selected from quaternary trialkylammonium, quaternary trialkylphosphonium, quaternary triarylphosphonium, mixed alkyl aryl. quaternary phosphonium groups, and ternary sulfonium groups.

19. The method of claim 13 wherein the nucleic acid binding groups are selected from quaternary trialkylammonium and quaternary trialkylphosphonium groups wherein the alkyl groups each have at least four carbon atoms, and wherein the nucleic acid binding groups cause lysis of cells and viruses to liberate ribonucleic acid.

20. The method of claim 6 wherein the solid phase binding materials comprise nucleic acid binding groups attached to a matrix through a selectively cleavable linkage.

21. The method of claim 1 wherein the acidic solution comprises an aqueous solution having a pH in the range of 1-5.

22. The method of claim 21 wherein the acidic solution comprises an aqueous solution having a pH in the range of 2-4.

23. The method of claim 21 wherein the acidic solution comprises an aqueous solution of an organic or inorganic acid selected from pyridinium salts, mineral acids, monocarboxylic acids, dicarboxylic acids, tricarboxylic acids, and amino acids, as well as their alkali metal, alkaline earth, transition metal, NH₄⁺, quaternary ammonium and quaternary phosphonium salts.

24. The method of claim 2 wherein the reagent for releasing bound ribonucleic acid from the solid phase comprises an alkaline solution having a concentration of alkali of 1 mM to 1 M.

25. The method of claim 1 wherein the solid phase material comprises magnetic particles having a tributylphosphonium nucleic acid binding group linked through a cleavable arylthioester linkage to a magnetic particle matrix.

26. The method of claim 25 wherein the solid phase material has the formula 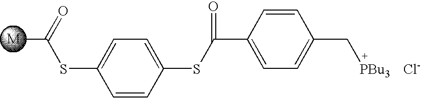
wherein 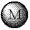
represents a silica-based magnetic particle functionalized with covalently attached linker groups.

27. The method of claim 1 wherein the biological sample is selected from bacterial cultures, pelleted cells from bacterial cultures, blood, blood plasma, blood serum, urine sputum, semen, CSF, plant cells, animal cells, and tissue homogenates.

28. A method for extracting ribonucleic acid from a biological sample selected from bacterial cultures, pelleted cells from bacterial cultures, blood, blood plasma, blood serum, urine sputum, semen, CSF, plant cells, animal cells, and tissue homogenates, the sample containing at least one of cells or viruses comprising:
a) contacting the sample with an acidic solution having a pH in the range of 1-5 wherein the acidic solution comprises an aqueous solution of an organic or inorganic acid selected from pyridinium salts, mineral acids, monocarboxylic acids, dicarboxylic acids, tricarboxylic acids, and amino acids, and their alkali metal, alkaline earth, zinc, NH₄⁺, quaternary ammonium and quaternary phosphonium salts to form a mixture;
b) combining the mixture with a solid phase binding material comprising a matrix portion and a nucleic acid binding portion wherein the solid phase binding material is selected to have the ability to liberate ribonucleic acid directly from biological samples without first performing any preliminary lysis, and wherein no chaotropic agents or detergents are used to effect lysis, and whereby the nucleic acid binding groups cause lysis of cells and viruses to liberate ribonucleic acid; and
c) binding ribonucleic acid on the solid phase.

29. The method of claim 28 wherein the solid phase material comprises magnetic particles having a tributylphosphonium nucleic acid binding group linked through a cleavable arylthioester linkage to a magnetic particle matrix.

30. The method of claim 29 wherein the solid phase material has the formula 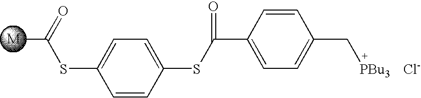
wherein 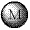
represents a silica-based magnetic particle functionalized with covalently attached linker groups.

31. The method of claim 30 further comprising:
d) separating the sample from the solid phase having ribonucleic acid bound thereto
e) optionally washing the solid phase with at least one wash solution; and
f) eluting the bound ribonucleic acid from the solid phase by contacting the solid phase material with a reagent comprising an alkaline solution having a concentration of alkali of 1 mM to 1 M to release the bound RNA into solution.

32. A method for isolating ribonucleic acid from a biological sample selected from bacterial cultures, pelleted cells from bacterial cultures, blood, blood plasma, blood serum, urine sputum, semen, CSF, plant cells, animal cells, and tissue homogenates, the sample containing at least one of cells or viruses comprising:
a) contacting the sample with an acidic solution having a pH in the range of 1-5, wherein the acidic solution comprises an aqueous solution of an organic or inorganic acid selected from pyridinium salts, mineral acids, monocarboxylic acids, dicarboxylic acids, tricarboxylic acids, and amino acids, and their alkali metal, alkaline earth, transition metal, NH₄⁺, quaternary ammonium and quaternary phosphonium salts to form a mixture;
b) combining the mixture with a solid phase binding material comprising magnetic particles having a tributylphosphonium nucleic acid binding group linked through a cleavable arylthioester linkage to a magnetic particle matrix wherein the solid phase binding material is selected to have the ability to liberate ribonucleic acid directly from biological samples without first performing any preliminary lysis, and wherein no chaotropic agents or detergents are used to effect lysis, and whereby the nucleic acid binding groups cause lysis of cells and viruses to liberate ribonucleic acid; and
c) binding ribonucleic acid on the solid phase;
d) separating the sample from the solid phase having ribonucleic acid bound thereto;
e) optionally washing the solid phase with at least one wash solution; and
f) cleaving the selectively cleavable linkage with a cleavage reagent thereby releasing the ribonucleic acid from the solid phase binding material.

33. The method of claim 32 wherein the cleavable linkage is selected from a hydrolytically cleavable group, a disulfide group, a peroxide bond, a group cleavable by an enzyme selected from esterases, hydrolases, proteases, peptidases, and glycosidases, a cleavable 1,2-dioxetane moiety, an electron-rich C—C double bond wherein the double bond is attached to at least one O, S, or N atom, a ketene dithioacetal compound, and a photocleavable linker group selected from nitro-substituted aromatic ethers and esters.

34. The method of claim 33 wherein the hydrolytically cleavable group is selected from carboxylic esters, carboxylic anhydrides, thioesters, carbonate esters, thiocarbonate esters, urethanes, imides, sulfonamides, sulfonimides and sulfonate esters.

35. The method of claim 34 wherein the hydrolytically cleavable linkage is cleaved by reaction with a reagent comprising an alkaline solution having a concentration of alkali of 1 mM to 1 M.
